(19) United States
(12) Patent Application Publication
(10) Pub. No.: US 2019/0224496 A1
(43) Pub. Date: Jul. 25, 2019

(54) WIRELESS SENSOR SYSTEM, FOR USE IN A PHOTOTHERAPY SYSTEM, CAPABLE OF HARVESTING ENERGY

(71) Applicant: KONINKLIJKE PHILIPS N.V., EINDHOVEN (NL)

(72) Inventors: Frank Anton VAN ABEELEN, Eindhoven (NL); Ernest Roderick LAMAN, Waalre (NL)

(21) Appl. No.: 16/337,167

(22) PCT Filed: Sep. 22, 2017
(86) PCT No.: PCT/EP2017/074011

§ 371 Date: Mar. 27, 2019

(30) Foreign Application Priority Data
Sep. 29, 2016 (EP) 16191292.8

(51) Int. Cl. A61N 5/06; A61B 5/145; A61B 5/1455; A61B 5/00; A61B 5/0205; A61B 5/01

(52) U.S. Cl. CPC A61N 5/0621; A61B 5/14546; A61B 5/1455; A61B 5/0002; A61B 5/02438; A61B 5/02055; A61B 5/01; A61B 5/14551; A61N 2005/0628; A61B 5/6833

(57) ABSTRACT

A sensor system, for use in a phototherapy system for treatment of neonatal jaundice, designed to harvest energy from the phototherapy system. The sensor system is wirelessly connected to the phototherapy system and provides measurements of the total serum bilirubin levels of the infant. The continuous measurement of the total serum bilirubin allows the phototherapy system to personalize the treatment of each infant, minimizing any unwanted side effects of the phototherapy. The energy harvesting performed by the sensor removes the need to manually replace or recharge batteries, which may disturb the infant during treatment, and may enable the sensor system to operate without an energy storage device, such as a battery, altogether.

### FIELD OF THE INVENTION

This invention relates to the field of phototherapy, and more specifically to neonatal phototherapy, for example to treat hyperbilirubinemia.

### BACKGROUND OF THE INVENTION

Neonatal jaundice is a condition that affects over half of all new-borns in their first week of life. In neonates, jaundice may develop because of two factors; the breakdown of fetal hemoglobin as it is replaced with adult hemoglobin and relatively immature hepatic metabolic pathways in the liver, which are unable to conjugate bilirubin and so excrete it as quickly as an adult. These cause an accumulation of bilirubin in the blood, known as hyperbilirubinemia, which leads to a visible yellowing of the skin.

High levels of bilirubin put the infant at risk of kernicterus, the deposit of toxic levels of bilirubin in the brain, which can cause permanent neurological impairment. It may take several days for the infant's system to begin eliminating bilirubin from the blood faster than it is produced. Bilirubin levels normally peak, in full-term infants, 4 to 5 days after birth and may peak later in premature infants. Treatment is required for 10-30% of full-term infants and 80% of pre-term infants.

Hyperbilirubinemia is treated by phototherapy, the irradiation of the body with blue light with a wavelength in the range of 460-490 nm. The light induces the conversion of bilirubin into lumirubin, which can be excreted by the body via the urine and feces without being processed by the liver. Phototherapy should be nearly continuous and may take several days.

Various phototherapy devices exist for treatment of neonatal jaundice, including; overhead lamps, under-the-bed lamps, 360° illumination capsules, pads with optical fibers illuminated from a source at the side and phototherapy blankets wherein, phototherapeutic light sources are integrated into a blanket. Each device possesses their own advantages and disadvantages, for example; a 360° illumination capsule may be able to deliver the highest dose of therapeutic light to the infant, a pad may be easily transported and stored when not in use. A phototherapy blanket possesses the advantages of; emitting less light into the surrounding environment, delivering a high dose of phototherapeutic light, allowing the mother to conveniently hold the baby, so that natural bonding may occur, and a more pleasant experience for the baby.

During phototherapeutic treatment the caregiver can check the infant's bilirubin level in order to monitor their response to the treatment. Dependent on the observed bilirubin level in a baby's blood, known as the total serum bilirubin (TSB) level, a neonatologist can prescribe different levels of phototherapy. Often treatment levels are described as single or double phototherapy, meaning irradiation from one side only (top or bottom) or irradiation from both sides, respectively. In very severe cases triple phototherapy may be prescribed, meaning either an increased brightness or an increased number of applied lamps. In most cases, the top irradiance is the one to be increased. During the treatment TSB is measured at regular intervals to confirm the decrease of bilirubin concentration and ultimately, to decide when phototherapy may be discontinued.

TSB can be measured from a blood sample or by a transcutaneous bilirubin measurement. A transcutaneous bilirubin measurement device has a source of white light which irradiates the skin of the patient. The light reflected from the skin is captured in a fiber optic bundle and taken to a micro-spectrometer within the device. The spectrometer determines the relative intensity of the returned light at a number of specific wavelengths. Based on knowledge of the spectral properties of components within the skin, it can subtract the interfering components and determine the concentration of bilirubin.

Exposure of a neonate to phototherapy may affect some of the interfering components within the skin. Therefore, for an accurate measurement of the TSB level, the measurement site should be covered so that light from the phototherapy device may not reach it. In current practice this is done with a small adhesive patch, such as a plaster.

In the past it has been accepted that the side effects of neonatal phototherapy are not serious and seem to be well controlled, however, recent research suggests that the exposure should be limited in order to minimize possible side effects of phototherapy.

It is currently common practice to administer an initial four to six hours of treatment and, after confirmation that the treatment has indeed reduced total serum bilirubin level, to check the total serum bilirubin level of an infant being treated every twelve hours in order to minimize discomfort for the infant. This means infants may be treated longer than necessary and thus suffer more side effects than necessary. In addition, it is often not possible for the neonatologist to dedicate more time to checking the TSB levels than once every twelve hours due to their large volume of work.

Document WO 2015/006656 discloses a method for monitoring a user's light exposure by receiving light exposure from a light source with an interactive light monitor. A photovoltaic cell converts the light exposure into electric current. At least some of the electric current is directed from the photovoltaic cell to an energy harvester, which is stepped-up and directed to an energy storage device.

Document US 2012/0199995 discloses an ophthalmic lens with a light source capable of providing specific bandwidths of light to an eye of a wearer. The lens may include a light source, light sensors and an energy source.

### SUMMARY OF THE INVENTION

The invention is defined by the claims.

According to examples in accordance with an aspect of the invention, there is provided a phototherapy system comprising:

a sensor system for a phototherapy system, comprising:
a sensor which comprises a light source and a light detector, for application to the skin;
a wireless communication unit for communicating sensor data; and
an energy harvesting device for harvesting energy received from the phototherapy device for powering the sensor and wireless communication unit; and

a controller for controlling the phototherapy light source.

This wireless arrangement allows the sensor system to be placed at any point on the infant's body without the risk of the infant becoming tangled in a wire during treatment. In addition, applying the sensor directly to the skin prevents the phototherapeutic light from reaching the measurement area of the sensor, leading to more accurate measurement results. The energy harvesting device within the sensor system allows for the sensor system to operate for long periods of time with only a small battery and eliminates the risk of the battery not being sufficiently charged before treatment commences, minimizing the discomfort caused to the infant by having to remove the sensor to replace the battery. This also increases the convenience of the system for the nurses caring for the infant as they do not have to spend time replacing the batteries. The sensor system may be used in phototherapy systems designed to treat a variety of conditions, such as neonatal jaundice or Crigler-Najjar.

The energy harvesting device is for obtaining electrical energy without the need for electrical connection, for example from an electromagnetic wave or field, or an electric or magnetic field.

In one arrangement, the sensor may have a skin contacting surface which comprises adhesive material. This means that the sensor may adhere to the skin of the infant, further reducing the risk of phototherapeutic light reaching the measurement area.

The sensor may be adapted to measure the total serum bilirubin level. The total serum bilirubin level is a key indicator of the severity of neonatal jaundice. By monitoring the total serum bilirubin levels, the phototherapy system or a user of the phototherapy system may assess how well the infant is responding to the treatment. This provides an accurate measure for the level of continued treatment required, limiting the unnecessary exposure of the infant to the phototherapeutic light.

The energy harvesting device may comprise a photovoltaic device for harvesting energy from phototherapy light. The phototherapeutic light that impacts the sensor is not used for phototherapy and instead enables energy harvesting in order to produce power for the sensor. This is advantageous as the sensor system is able to operate for a longer period of time with a smaller battery than would otherwise be required and eliminates the risk of the battery not being sufficiently charged before treatment commences, meaning that the infant receives less interruption due to battery changes.

The sensor system may comprise a housing, wherein the sensor is provided at one location of the housing over a sensor aperture, and the photovoltaic device is provided at another location of the housing. The housing may be designed to have a low profile so as to minimize discomfort to the infant. Furthermore, the housing may be partially constructed from a flexible material so as to not constrict the movement of the infant, further reducing discomfort caused by the sensor.

The photovoltaic device may be adapted for converting phototherapy light with a wavelength in the range of 460-490 nm. Blue light with a wavelength in the range of 460-490 nm is the light primarily used in phototherapeutic treatment of neonatal jaundice as it induces the conversion of bilirubin inside the blood into lumirubin, which may be excreted without processing by the liver.

The phototherapy device may further comprise a transmitter inductor coil, wherein the energy harvesting device comprises a receiver inductor coil for harvesting energy by inductive energy transfer from the transmitter inductor coil. The transmitter coil may be large so as to ensure that the receiver coil remains within the coupling boundaries of the transmitter coil, regardless of the position of the sensor on the infant's body.

The electromagnetic coupling between the phototherapy system and the energy harvesting device may be weak, however, the sensor system possesses a low energy requirement.

In some other designs, the phototherapy device may further comprise a second light source, wherein the energy harvesting device is for harvesting energy from the light output of the second light source. The second light source may comprise a visible or infrared light source. This arrangement allows the phototherapeutic light source to operate at a lower power level or for the phototherapy device to operate in single phototherapy mode, meaning that the infant is only illuminated from one side, for less severe cases of jaundice. This allows for the sensor to be placed in a comfortable location for the infant that may not receive a high level of phototherapeutic light without depriving the sensor of an energy source.

In some arrangements, the controller may be adapted to adjust the phototherapy settings in dependence on the sensor system output. This self-regulation by the phototherapy system removes the need for constant monitoring and adjustment of the system by the carer of the infant.

According to examples in accordance with an aspect of the invention, there is provided a sensing method, comprising:

harvesting external energy received from a phototherapy system;

powering a sensor and a wireless communication unit using the harvested energy, wherein the sensor comprises a light source and a light detector, for application to the skin.

In some embodiments the method for harvesting external energy comprises:

harvesting energy from phototherapy light of the phototherapy system; or

harvesting energy from light provided by the phototherapy system in addition to phototherapy light; or

harvesting energy from the phototherapy system by wireless inductive energy transfer.

### BRIEF DESCRIPTION OF THE DRAWINGS

Examples of the invention will now be described in detail with reference to the accompanying drawings, in which:

FIG. 1 shows a sensor system;

FIG. 2 shows an embodiment of the system shown in FIG. 1;

FIG. 3 shows a further embodiment of the system shown in FIG. 1;

FIG. 4 shows a phototherapy system comprising the sensor system shown in FIG. 1;

FIG. 5 shows an embodiment of the system shown in FIG. 4 comprising a second light source;

FIG. 6 shows a further embodiment of the system shown in FIG. 4 comprising an inductive coil;

FIG. 7 shows an application of the system shown in FIG. 4, wherein the phototherapy device shown is a phototherapy blanket; and

FIG. 8 shows a method of the invention.

### DETAILED DESCRIPTION OF THE EMBODIMENTS

The invention provides a sensor system for a phototherapy system which includes a sensor, comprising a light source and a light detector, a wireless communication unit and an energy harvesting device for harvesting energy received from the phototherapy device.

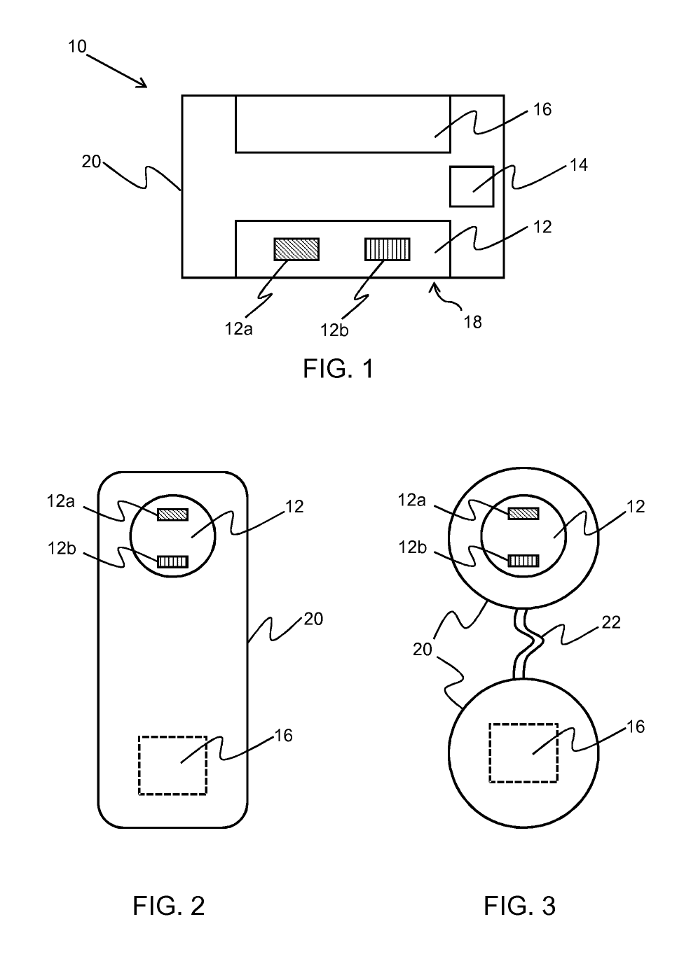

FIG. 1 shows a sensor system 10 which comprises a sensor 12, which comprises a light source 12a and a light detector 12b, for application to the skin, a wireless communication unit 14 for communicating sensor data and an energy harvesting device 16 for harvesting external energy received from the phototherapy system for powering the sensor and wireless communication unit.

The sensor system 10 may have a skin contacting surface 18 which comprises an adhesive surface and may be adapted to measure the total serum bilirubin level.

When treating a condition such as neonatal jaundice, a key indicator of the effectiveness of the treatment is the level of bilirubin in the infant's blood, known as total serum bilirubin (TSB). Using a wireless sensor to measure the TSB allows for the sensor to be placed at any point on the infant's body, minimizing the discomfort to the infant and eliminating the risk that the infant will become tangled in wires during treatment. Adapting the sensor system to include an energy harvesting device, to harvest energy from the phototherapy system, allows the sensor system to operate for a long period of time without needing to replace or recharge the batteries. In some cases, the sensor system may be able to operate without an energy storage device, such as a battery.

In addition to the TSB sensor, the sensor system may include sensors for vital signs, such as body temperature, heart rate and SpO2.

In order to achieve an accurate measurement of the TSB, the measurement site must be covered in order to prevent contact by the phototherapeutic light. TSB is measured by irradiating the skin of the patient with white light and then collecting the light reflected by the skin, which is then analyzed by a spectrometer. By subtracting the interfering components, based on known spectral properties of the skin, from the reflected light the TSB can be measured. Phototherapeutic light is known to alter several of the known spectral properties of the skin, leading to less accurate measurements of the TSB if the measurement site is exposed. By ensuring that the sensor 12 is in contact with the skin, the reflected light can be effectively collected without the phototherapeutic light contacting the measurement site and interfering with the TSB measurements. This risk is further minimized if the skin contacting surface 18 comprises an adhesive material, fixing the sensor to the infant's skin. The sensor system may also be attached to the infant's skin by way of a disposable plaster, surgical tape or the like.

The energy harvesting device 16 used to harvest energy from the phototherapeutic system may comprise a photovoltaic device. The photovoltaic device may contain a photodiode operated in photovoltaic mode. In other words, the energy harvesting device may be adapted to convert the phototherapy light into electric energy. The phototherapy light may have a wavelength in the range of 460-490 nm.

Phototherapeutic light is abundant in this system and so adapting the energy harvesting device 16 to harvest energy from the phototherapeutic light enables the sensor system to be used at any point within the phototherapy system. In addition, the phototherapy system does not have to supply additional power to the sensor system as the phototherapeutic light will always be operational during treatment thereby providing the power source for the sensor. This gives an energy efficient system. Phototherapeutic light, for treating neonatal jaundice, commonly possesses a wavelength in the range of 460-490 nm. This blue light induces the conversion of bilirubin into lumirubin which may be excreted by the body without needing to be processed by the liver. Adapting the energy harvesting device 16 to harness light of this wavelength will maximize the efficiency of the sensor system 10.

The sensor system may comprise a housing 20, wherein the sensor 12 is provided at one location over a sensor aperture, and the energy harvesting device 16, for example a photovoltaic device, is provided at another location of the housing. The housing may be small so as to minimize discomfort to the infant. Further examples of housing designs are shown in FIGS. 2 and 3.

FIG. 2 shows an embodiment of the sensor system shown in FIG. 1, wherein the sensor 12 is provided on one face of the housing 20 and the energy harvesting device 16 is provided on the opposing face of the housing.

In this design, the housing 20 has been elongated in order to spatially separate the sensor 12 and the energy harvesting device 16. This enables the sensor and energy harvesting device to be positioned at separate points on the infant's body to allow for optimal positioning of the sensor whilst maintaining sufficient exposure of the energy harvesting device to the energy source. The housing 20 may be partially constructed from a flexible material so as to not restrict the movement of the infant and to minimize discomfort. An example of this would be during single phototherapy where the infant is illuminated by a single light source, shining on its back. The sensor section may need to be placed on the infant's chest due to a variety of reasons or limitations. As the energy harvesting device is spatially separated from the sensor, it may be fixed close to the infant's back so as to receive sufficient exposure to the light. The flexible material allows for the housing to shape itself to the infant, who otherwise may be restricted to an uncomfortable position.

FIG. 3 shows a further embodiment of the sensor system shown in FIG. 1, wherein the sensor 12 and the energy harvesting device 16 are provided in individual housings 20, with a connection 22 connecting the housings.

The advantages of this embodiment are much the same as in the embodiment shown in FIG. 2. However, by removing unnecessary housing material, the infant may be able to enjoy a greater range of movement without experiencing discomfort form the sensor.

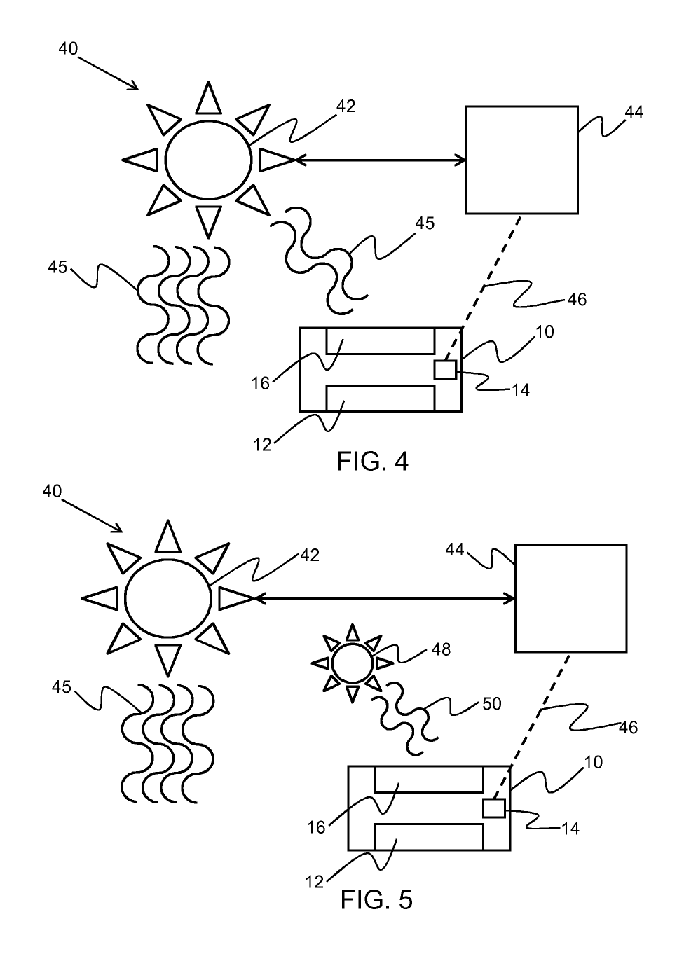

FIG. 4 shows a phototherapy system 40 comprising a light source 42, a controller 44 for controlling the light source and a sensor system 10 as shown in FIG. 1. The light source 12a and light detector 12b of the sensor 12 have been omitted from FIGS. 4 to 7 for clarity. The light source produces phototherapeutic light 45, for treating the patient, which may be harvested by the energy harvesting device 16 in order to power the sensor. There is a wireless connection 46 between the sensor and the controller.

In this embodiment, the sensor system 10 may harvest energy from the phototherapeutic light 45 that is abundant throughout the phototherapy system and does not require an additional source to harvest energy from. The wireless connection 45 between the controller 44 and the sensor system means that the placement of the sensor system is not restricted by wires and the risk of the infant becoming tangled in said wires is eliminated. In addition, the wireless connection between the sensor system and controller allows the sensor system to communicate sensor data to the controller. The controller may then use this data to evaluate the level of treatment needed by the patient.

By harvesting energy from the phototherapy system, the sensor does not need to be exposed to external light, for example, and can thus be fully contained within the phototherapy system, for example blanket.

If the data returned by the sensor system indicates that the TSB is above a predetermined threshold, the controller may increase the intensity of the light source 42 or activate additional phototherapeutic light sources, known as double, or triple, phototherapy. If the data indicates that the TSB is below a predetermined threshold, the controller may decrease the intensity of the light source or deactivate additional light sources that were previously active, returning to a single, or double, phototherapy mode. A predetermined threshold may be used to switch off the phototherapy light source completely when treatment is no longer necessary. The light source may also operate on a duty cycle, meaning that it may be active for a given percentage of the treatment time and inactive for the remainder. The duty cycle may be altered by the controller in a similar manner to the intensity. A combination of these methods means that therapeutic effectiveness is maintained by the system whilst minimizing unnecessary side effects. The initial level of treatment may be automatically selected by the system at the beginning of the therapy based on the measured TSB level.

In some embodiments, the controller 44 may also comprise a user interface for informing the caregiver of the TSB measurements or for allowing the caregiver to manually control the level of treatment provided by the phototherapy system. In a further embodiment, the controller may communicate with a separate user interface such as a bedside patient monitor, a tablet computer, a smartphone, a computer in a nurse room as used in a neonatal intensive care unit or any other connected device of the caregiver in order to perform these functions at a remote location.

The user interface may provide information to the caregiver, such as the current TSB level, the TSB level trend and/or the TSB level as a function of time. The trend of the TSB level could be shown as increasing or decreasing and may be combined with an indication of the speed at which it is increasing or decreasing. The user interface may also display a recommendation to the caregiver to change the settings of the phototherapy device in order to optimize treatment. This recommendation may be accompanied by a visible or audible alarm intended to alert the user that more preferable settings are available.

FIG. 5 shows an alternative embodiment of the system shown in FIG. 4, wherein the phototherapy system 40 now comprises a second light source 48 that produces light 50 to be harnessed by the energy harvesting device 16 in the sensor system 10. In some designs, the second light source may produce light with a wavelength in the visible or infrared ranges.

In this arrangement, the energy harvesting device 16 may harvest energy to power the sensor system 10 from the second light source 48. The second light source may be arranged so that the intensity of the light 50 is not affected by the data supplied to the controller 44 from the sensor system. This prevents the sensor system from losing power from insufficient light exposure and it may operate regardless of the level of treatment being given or the area of the body being treated.

In some or other embodiments, the energy harvesting device 16 may harvest energy from both the phototherapy light 45 and the light 50 produced by the second light source 48.

FIG. 6 shows another alternative embodiment of the system shown in FIG. 4, wherein the phototherapy system 40 comprises a transmitter inductor coil 52. The energy harvesting device 16 may comprise a receiver inductor coil for harvesting energy by inductive energy transfer 54 from the transmitter inductor coil.

An alternating electromagnetic field is established by the transmitter inductor coil. Electromagnetic coupling with the receiver inductor coil enables energy transfer between the coils.

In this design, the transmitter inductor coil 52 may be large so as to ensure that the transmitter inductor coil and the receiver inductor coil may be inductively coupled when the sensor system 10 is placed at any point in the phototherapy system 40, or at any point within an inductive coupling region associated with the transmitter coil. The inductive coupling between the transmitter inductor coil and the receiver inductor coil may be weak; however, the sensor system does not require a large amount of power to operate.

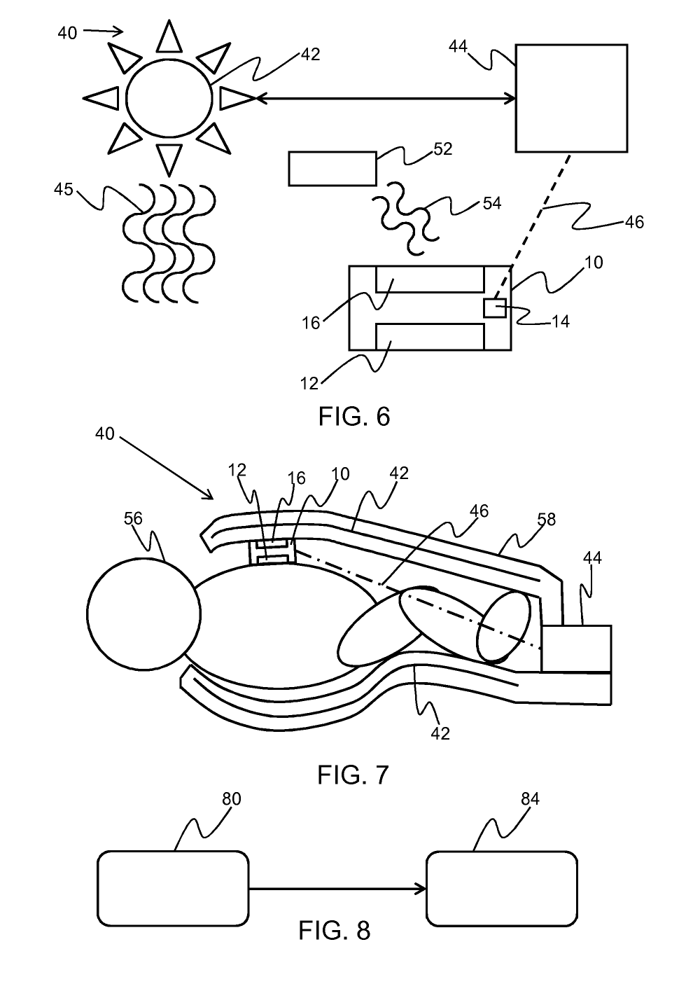

FIG. 7 shows a phototherapy system 40 for treating an infant 56 suffering from hyperbilirubinemia. In the embodiment shown, the phototherapy system is a phototherapy blanket 58. The sensor system 10 is attached to the infant's skin so that the sensor 12 is facing the infant and the energy harvesting device 16 is facing a phototherapy light source 42. In this example the energy harvesting device is a photovoltaic device and the phototherapy light source is an LED array within the blanket. There is a wireless connection 46 between the sensor system and the controller 44 for controlling the light source.

The wireless connection 46 allows the sensor system to communicate sensor data to the controller, which may use the data to decide the level of treatment necessary for the infant and adjust the light source 42 accordingly. In addition, the wireless connection allows the infant to move freely within the blanket without the danger of becoming tangled in wires. Attaching the sensor to the skin of the infant means that the TSB levels measured by the sensor system will be more accurate. This means that the infant will receive the correct level of treatment, minimizing unnecessary side effects.

In further embodiments, the phototherapy system 40 may be adapted in a similar manner to the systems shown in FIGS. 5 and 6. The LED arrays held by the blanket may be adapted to provide light in the visible or infrared spectrum for the purpose of being converted to electrical energy by the energy harvesting device 16. In the case of inductive coupling, the transmitter inductor coil may be woven into the fabric of the phototherapy blanket. This arrangement would ensure that the receiver inductor coil within the energy harvesting device would be able to inductively couple with the transmitter inductor coil from any point on the infant's body.

FIG. 8 shows a method of the invention.

In step 80, the sensor system harvests energy from the phototherapy system through an energy harvesting device.

In step 84, the harvested energy is used to power a sensor and a wireless communications unit. The sensor comprises a light source and a light detector, for application to the skin, and the wireless communications unit is for communicating the sensor data.

The sensor information may then be used to control the phototherapy system in order to deliver an optimum phototherapy treatment.

The energy harvesting of step 80 may comprise harvesting energy from phototherapy light of the phototherapy system, or from additional light provided by the phototherapy system in addition to the phototherapy light, or using wireless inductive energy transfer.

The energy harvesting in the examples above is based on electromagnetic waves or fields. In further embodiments, energy harvesting may also be performed through wireless transmission methods other than resonant inductive coupling or photoelectric harvesting (as described above), such as capacitive coupling, microwave radiation or the like. However, other energy transfer methods may be used which do not require electromagnetic interaction. These may be based on thermal, mechanical or electrical properties. Some methods may require physical (but not electrical) contact and others enable transmission through air and therefore enable more freedom in the spacing of components. All of these options are possible and intended to fall within the broad term “external energy”.

For example, the energy harvesting may be performed by a piezoelectric device, a thermoelectric device, a pyroelectric device or the like.

The sensor in the examples above is adapted to measure a total serum bilirubin (TSB) level of a patient, such as an infant. However, the sensor may be adapted to measure alternate, or additional, indicators from a patient's skin.

For example, in a phototherapy system adapted to treat neonatal jaundice, as described above, the sensor may be adapted to measure the melanin content of the skin in addition, or alternatively, to the TSB level. In this way, it is possible to monitor a change in melanin level due to phototherapeutic treatment, thereby allowing the caregiver, or phototherapy system, to adjust the phototherapy device to prevent the over production of melanin in the infant.

Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

1. A phototherapy system comprising:
a phototherapy device comprising a phototherapy light source:
a sensor system for a phototherapy system, comprising:
a Total serum bilirubin (TSB) sensor for contacting with the skin, the sensor comprising a light source and a light detector;
a wireless communication unit for communicating sensor data; and
an energy harvesting device for harvesting energy received from the phototherapy device for powering the sensor and wireless communication unit; and
a controller for controlling the phototherapy light source.

2. A system as claimed in claim 1, wherein the sensor system has a skin contacting surface which comprises adhesive material.

3. A system as claimed in claim 1, wherein the sensor system further comprises at least one sensor for measuring a vital sign, preferably, body temperature, heart rate and SpO2.

4. A system as claimed in claim 1, wherein the energy harvesting device comprises a photovoltaic device for harvesting energy from the light output of the phototherapy light source.

5. A system as claimed in claim 4, comprising a housing wherein the sensor is provided at one location of the housing over a sensor aperture, and the photovoltaic device is provided at another location of the housing.

6. A system as claimed in claim 4, wherein the photovoltaic device is for converting phototherapy light with a wavelength in the range of 460-490 nm.

7. A system as claimed in claim 1, wherein the phototherapy device further comprises a transmitter inductor coil, wherein the energy harvesting device comprises a receiver inductor coil for harvesting energy by inductive energy transfer from the transmitter inductor coil.

8. A system as claimed in claim 1, wherein the phototherapy device further comprises a second light source, wherein the energy harvesting device is for harvesting energy from the light output of the second light source.

9. A system as claimed in claim 8, wherein the second light source comprises a visible or infrared light source.

10. A system as claimed in claim 1, wherein the controller is adapted to adjust the phototherapy settings in dependence on the sensor system output.

11. A sensing method, comprising:
harvesting external energy received from a phototherapy system;
powering a sensor and a wireless communication unit using the harvested energy, wherein the sensor comprises a Total serum bilirubin (TSB) sensor for contacting with the skin and wherein the sensor comprises a light source and a light detector.

12. A method as claimed in claim 11, wherein harvesting external energy comprises:
harvesting energy from phototherapy light of the phototherapy system; or
harvesting energy from light provided by the phototherapy system in addition to phototherapy light; or
harvesting energy from the phototherapy system by wireless inductive energy transfer.
